(19) United States
(12) Patent Application Publication
(10) Pub. No.: US 2015/0330935 A1
(43) Pub. Date: Nov. 19, 2015

(54) TEST DEVICE FOR ELECTROCHEMICAL ANALYSIS

(71) Applicant: ALERE SWITZERLAND GMBH, Zug (CH)

(72) Inventors: Manus Joseph DENNISON, Alloa (GB); Murray John WHYTE, Devonside Tillicoultry (GB); Christopher John SLEVIN, Edinburgh (GB); Anthony BOYLAN, Menstrie (GB); David William TAYLOR, Alloa (GB); John Anthony BOLBOT, Alva (GB); Jerome MCALEER, Minster Lovell Oxfordshire (GB)

(21) Appl. No.: 14/654,490

(22) PCT Filed: Dec. 20, 2013
(86) PCT No.: PCT/EP2013/077797

§ 371 Date: Jun. 19, 2015

Related U.S. Application Data
(60) Provisional application No. 61/745,211, filed on Dec. 21, 2012.
(60) Provisional application No. 61/770,233, filed on Feb. 27, 2013.

(30) Foreign Application Priority Data
Dec. 21, 2012 (GB) 1223166.8

(51) Int. Cl. G01N 27/327; G01N 33/561; C07D 417/12; C07D 279/18; C07D 279/36

(52) U.S. Cl. CPC G01N 27/3272; C07D 279/18; C07D 279/36; C07D 417/12; G01N 33/561

(57) ABSTRACT

The present invention relates to test devices for determining the presence of one or more analytes in a sample, methods for using such test devices and methods of manufacturing such test devices. The test devices comprise a substrate having disposed thereon, two or more conductive tracks, a reagent composition and a top layer covering a portion of the conductive tracks which forms, in combination with the substrate, a sample receiving chamber. At least one of the conductive tracks comprises a conductive polymer.

### CROSS-REFERENCE TO RELATED APPLICATIONS

The present invention is filed under 35 U.S.C. §371 as the U.S. national phase of international application No. PCT/EP2013/077797, filed Dec. 20, 2013, which designated the U.S. and claims the benefit of priority to GB Patent Application No. 1223166.8 filed Dec. 21, 2012, and to U.S. Provisional Application No. 61/745,211, filed Dec. 21, 2012, and to U.S. Provisional Application No. 61/770,233, filed Feb. 27, 2013, the contents of each of which is hereby incorporated by reference in its entirety for all purposes.

### FIELD OF THE INVENTION

The present invention relates to test devices for determining the presence of one or more analytes in a sample, methods for using such test devices, and methods of manufacturing such test devices.

### BACKGROUND

Test strips including conductive tracks are used to determine the presence or amount of an analyte, such as an enzyme substrate, in a fluid sample. A meter or reader is used in conjunction with a test strip to perform an electrochemical measurement on a sample applied to a test strip to provide an assay result.

### SUMMARY

In a first aspect, the invention provides a test device comprising;
a substrate having disposed thereon two or more conductive tracks;
a reagent composition disposed over a portion of at least one conductive track; and
a top layer covering a portion of the two or more conductive tracks which forms in combination with the substrate a sample receiving chamber;
   wherein at least one conductive track comprises a conductive polymer.

The two or more conductive tracks may include first and second ends, wherein the first end is at a distal end of the device and the second end is at a proximal end of the device. The distal end of the test device may be for engagement with an instrument configured to receive and/or supply electrical signal such as a test meter and may include two or more contacts. The sample receiving chamber may be located at the proximal end of the device. Each conductive track may comprise an electrode. The electrode may be located at the proximal end of the device. Preferably, the test device is a test strip, more preferably a disposable test strip, for determining the presence of one or more analytes in a sample. Preferably, the substrate is an insulating substrate and preferably comprises or consists of polyester, polycarbonate, polystyrene, polymethylmethacrylate, or combinations thereof. The substrate may have a length L1 and a width W2, first and second major surfaces and a distal end and a proximal end. In some embodiments, the test device comprises an insulating layer applied over at least a portion of the conducting polymer to define an area of the conductive polymer that may be exposed to a sample.

The test device may comprise two, three, four, five, six or more conductive tracks. Each track may be similar or identical in length, width, thickness and/or two-dimensional shape to other conductive tracks or may have a different length, width and/or two-dimensional shape. Each track may comprise or consist of a conductive polymer and/or a conductive material other than a conductive polymer, with the proviso that at least a portion of one track of the device comprises a conductive polymer.

Each conductive track may have a length Lₜ of at least about 25 mm on a major surface of the insulating substrate. In some embodiments, a length L_{cp} of at least about 5 mm, 7.5 mm, 10 mm, 12.5 mm, 15 mm, 17.5 mm, 20 mm or at least about 25 mm of the conductive track is formed of conductive polymer. The length Lₜ may be equal to length L_{cp}. Each conductive track may comprise an electrode which may comprise or consist of conductive polymer. The device may comprise two, three, four, five, six or more electrodes. A conductive track may form at least a “working electrode” or “measurement electrode”, a “counter electrode” or a “reference electrode”.

The test device may have multiple working electrodes, counter electrodes and/or reference electrodes. Preferably, one or more conductive tracks of the device are configured to pass electrical signal to an instrument capable of receiving and/or sending electrical signals. Preferably, the instrument is a test meter such as a glucose meter.

In some embodiments, a length L_{cm} of at least about 20 mm of at least one conductive track is formed of conductive material. In other embodiments, the length L_{cm} may be less than about 17.5 mm, less than about 15 mm, less than about 12.5 mm, less than about 10 mm, less than about 7.5 mm, less than about 5 mm, less than about 2.5 mm, or less than about 0.1 mm.

In some embodiments, the device is provided with at least one “narrow” conductive track, in electrical communication with at least one of the at least two conductive tracks of the device, preferably a measurement electrode and/or a counter electrode. At least a portion of the narrow conductive track has a width less than the width of other conductive tracks present in the device and is configured to provide an electrical signal to a microprocessor of an instrument such as a test meter. This allows the microprocessor to determine the voltage present at an adjacent conductive track, preferably the measurement electrode and/or the counter electrode. Preferably, at least a portion of the narrow conductive track and in some embodiments, the entire narrow conductive track, has a width of less than about 1 mm, more preferably less than or equal to 500 μm, less than or equal to 250 μm, less than or equal to 100 μm, less than or equal to 75 μm, less than or equal to 50 μm, less than or equal to 25 μm or less than or equal to 10 μm. At least a portion of the narrow conductive track may have a width of 50% or less, 25% or less, 10% or less, 5% or less or 1% or less than all other conductive tracks present in the device. Other conductive tracks in the device typically have a minimum width of at least about 1 mm.

The conductive polymer may comprise polythiophene, polypyrrole, polyaniline, polyfluorene, polyacetylene, poly(p-phenylene vinylene), poly(3,4-ethylenedioxythiophene), poly(3,4-propylenedioxythiophene) and poly(3,3-dibenzyl-3,4-propylenedioxythiophene), poly(3-4-ethylenedioxythiophene), bis-poly(ethyleneglycol), lauryl terminated, poly(3,4-ethylenedioxythiophene)-block PEG, poly(3,4-ethylenedioxythiophene), tetramethacrylate end-capped or combinations thereof. For example, the conductive polymer may comprise a complex comprising a polymer disclosed herein and a counterion. In one embodiment, the conductive polymer is a complex comprising poly(3,4-ethylenedioxythiophene) and a counterion. The counterion may be polystyrene sulfonate (PSS), perchlorate, perchlorate p-toluene, sulfonate p-toluene or tosylate. In a preferred embodiment, the conductive polymer is a complex comprising poly(3,4-ethylenedioxythiophene) and polystyrene sulfonate (PEDOT:PSS).

In certain embodiments, the conductive polymer is modified to include functional reactive groups for attachment of an enzyme or a mediator. In other embodiments a linker molecule, such as a carbonyl linker molecule, is used to tether a mediator or an enzyme to the conducting polymer. When the linker is used to tether a mediator, the linker preferably provides for migration of the mediator between the active site of an enzyme and an electrode surface, thereby facilitating the transfer of electrons from the enzyme to the electrode.

The present inventors have found that devices having conductive tracks comprising or consisting essentially of conductive polymers have several advantages over known devices which employ other materials such as carbon or gold to form electrodes/conductive tracks. For example, conductive polymers allow a much higher level of batch to batch consistency to be achieved due to a reduced coefficient of variation associated with conductive polymers as compared to, for example, carbon. Consistency between batches of devices means that a single, universal calibration can be applied to all devices. This avoids the need for end users to calibrate the device which can lead to significant errors in test results. Conductive polymers used in the present invention are also significantly less expensive than other materials commonly used in known test devices such as gold. The reduced cost of each test device relative to known test devices means that it is feasible for test devices of the invention to be “single-use” and disposable.

Conductive tracks of the present invention may comprise a conductive material that is not a conductive polymer, such as a conductive material selected from the group comprising carbon, gold, platinum, silver, palladium, copper, indium tin oxide and combinations thereof.

The reagent composition may be provided in contact with the at least two conductive tracks. In some embodiments, the reagent composition is disposed within the sample receiving chamber and may cover all exposed conductive tracks and substrate therein. The reagent composition preferably includes an oxidoreductase enzyme and a mediator compound. The oxidoreductase enzyme may be selected from the group consisting of glucose oxidase, glucose dehydrogenase, lactate dehydrogenase, alcohol dehydrogenase, hydroxybutyrate dehydrogenase, cholesterol oxidase, amino acid oxidase, pyruvate oxidase, peroxidase, sarcosine oxidase, lactate oxidase, alcohol oxidase, monoamine oxidase, glycerol oxidase, glycerol phosphate oxidase, urate oxidase, xanthine oxidase, ascorbate oxidase, catalase and diaphorase.

Preferably, the oxidoreductase is glucose oxidase or glucose dehydrogenase. The glucose dehydrogense may be selected from a quinoprotein glucose dehydrogenase, a FAD dependent glucose dehydrogenase, and a NAD dependent glucose dehydrogenase. In certain embodiments the glucose dehydrogenase is FAD dependent Glucose Dehydrogenase from Sekisui Diagnostics, Catalogue Number GLDE-70-1192 (E.C. number 1.1.99.10) from Aspergillus sp. or FAD dependent Glucose Dehydrogenase from BBI enzymes, Catalogue Number GLD1.

The mediator compound may be selected from the group including potassium ferricyanide, ferrocene derivatives, phenoxazine derivatives, phenothiazine derivatives, quinone derivatives, and reversible redox transition metal complexes, particularly those of Ruthenium and Osmium, nicotinamide adenine dinucleotide (phosphate), diimines, phenanthroline derivatives, dichlorophenolindophenol tetrazolium dyes, and phenylimino-benzophenoxazine. In certain specific embodiments the mediator compound is 3-(3′,5′-dicarboxy-phenylimino)-3H-phenothiazine. Any other mediator compound disclosed herein can be used in the devices and methods of the present invention, either as the sole mediator compound or in combination with any other mediator compound disclosed herein.

In some embodiments the oxidoreductase enzyme and/or the mediator compound may be incorporated within or attached to the conducting polymer by way of chemical bonding or physical entrapment.

The invention also provides a test strip comprising; a substrate, which may be an insulating substrate; and a conductor supported by the insulating substrate, the conductor extending from at least a reagent test zone of the test strip to a second portion of the test strip operatively separated from the reagent test zone, wherein the portion of the conductor that is operatively separated from the reagent test zone comprises a conductive polymer.

Also provided is a test strip including a conductive polymer on an insulating substrate, where the conductive polymer defines at least a portion of at least one conductive track that carries an electrical signal from a meter or reader to a measurement electrode on the test strip. The conductive polymer may form at least the measurement electrode. The conductive polymer may form the entire conductive track on the test strip.

Also provided is a device, comprising;

an insulating substrate;
a conductive track with a length Lₜ of at least about 25 mm on a major surface of the insulating substrate, the conductive track comprising at least one electrode;
wherein a length L_{cp} of at least about 5 mm of the conductive track is formed of conductive polymer, and a length L_{cm} of at least about 20 mm of the conductive track is formed of conductive material.

In a second aspect, a method of manufacturing a test device is provided, the method comprising: forming a layer of conductive polymer, wherein at least one of the tracks comprises a conductive polymer; defining at least two electrically insulated conductive tracks; applying a reagent composition over a portion of at least one of the tracks; and forming a sample receiving chamber over the reagent composition, and a portion of at least one of the tracks.

The step of forming a layer of conductive polymer or conductive material may comprise applying the conductive polymer or conductive material to a substrate, preferably an insulating substrate. Preferably, the substrate forms part of the test device. The step of defining the electrically insulated tracks can be concurrent with the step of forming a layer of conductive polymer. For example, screen printing, gravure printing, or ink-jet printing may be used to deposit a conductive polymer or conductive material to define at least two electrically insulated conductive tracks. Alternatively, a layer of the conductive polymer may be formed, for example by coating an insulating substrate with the conductive polymer, and subsequently patterned by a process of laser ablation to define the at least two electrically insulated conductive tracks. Conductive material other than conductive polymer can be applied to the test device in the same way that the conductive polymer is applied. In some embodiments, an insulating layer is applied over at least a portion of the conductive polymer and/or the conductive material to define an area of the conductive polymer and/or conductive material that is for exposure to a sample. This method can be used to manufacture the devices according to the first aspect of the invention.

In a third aspect, the present invention provides a method comprising: contacting (a) a sample comprising a bodily fluid and (b) a reagent configured to facilitate detection of an analyte in the bodily fluid with a first electrode; and passing a first electrical signal from the first electrode along a first conductor in electrical communication with the first electrode, at least a portion of the first conductor being spaced apart from the first electrode in contact with the bodily fluid and reagent, wherein the first conductor comprises a conductive polymer.

In some embodiments, the first conductor consists essentially of a conductive polymer. Passing the electrical signal may include conducting the electrical signal along a length of the first conductor that consists essentially of the conductive polymer. The electrical signal may be passed along a length of the first conductor of at least about 5 mm, at least about 7.5 mm, at least about 10 mm, or at least about 12.5 mm. The first electrode may comprise a conductive polymer or may be formed from a conductive polymer, which may be the same as the conductive polymer of the first conductor.

The step of contacting may further comprise contacting (a) the sample comprising the bodily fluid and (b) the reagent configured to facilitate detection of an analyte in the bodily fluid with a second electrode, the second electrode being spaced apart from the first electrode; and passing a second electrical signal along a second conductor, the second conductor being in electrical communication with the second electrode and spaced apart from the first conductor and the first electrode. The second electrical signal may be passed from the second electrode along the second conductor.

The second conductor may comprise or consist essentially of a conductive polymer in electrical communication with the second electrode. The second electrode may comprise a conductive polymer or may be formed from a conductive polymer, which may be the same as the conductive polymer of the second conductor. The second electrical signal may be passed along a portion of the second conductor that consists essentially of the conductive polymer.

The second electrical signal may be passed along a length of conductive polymer of the second conductor of at least about 5 mm, at least about 7.5 mm, at least about 10 mm, or at least about 12.5 mm.

The step of contacting may further include contacting (a) the sample comprising the bodily fluid and (b) the reagent configured to facilitate detection of an analyte in the bodily fluid with a third electrode, the third electrode being spaced apart from the first and second electrodes; and passing a third electrical signal along a third conductor, the third conductor being in electrical communication with the third electrode and spaced apart from the first and second conductors and the first and second electrodes. The third electrical signal may be passed from the third electrode along the third conductor.

The third conductor may comprise or consist essentially of a conductive polymer in electrical communication with the third electrode. The third electrode may comprise a conductive polymer or may be formed from a conductive polymer, which may be the same as the conductive polymer of the third conductor.

Passing the third electrical signal may include passing the third electrical signal along a portion of the third conductor that consists essentially of the conductive polymer.

Passing the third electrical signal may include passing the third electrical signal along a length of conductive polymer of the third conductor of at least about 5 mm, at least about 7.5 mm, at least about 10 mm, or at least about 12.5 mm.

The method may further include passing the electrical signal passed along the first conductor to a first contact of an instrument configured to receive and/or supply the electrical signal. The method may further include mechanically engaging the first conductor with the first contact.

In some embodiments, the method includes (a) passing the electrical signal passed along the first conductor to a first contact of an instrument configured to receive and/or supply the electrical signal and (b) passing the electrical signal passed along the second conductor to a second contact of the instrument configured to receive and/or supply the electrical signal.

The first conductor may be mechanically engaged with the first contact and the second conductor may be engaged with the second contact. Engaging the second conductor with the second contact may include mechanically engaging conductive polymer of the second conductor in electrical communication with the second electrode with the second contact.

In some embodiments, the method includes (a) passing the electrical signal passed along the first conductor to a first contact of an instrument configured to receive and/or supply the electrical signal, (b) passing the electrical signal passed along the second conductor to a second contact of the instrument configured to receive and/or supply the electrical signal, and (c) passing the electrical signal passed along the third conductor to a third contact of the instrument configured to receive and/or supply the electrical signal.

The first conductor may be mechanically engaged with the first contact, the second conductor may be engaged with the second contact, and the third conductor may be engaged with the third contact.

Engaging the second conductor with the second contact may include mechanically engaging the second conductor, preferably conductive polymer of the second conductor in electrical communication with the second electrode, with the second contact. Engaging the third conductor with the third contact may include mechanically engaging the third conductor, preferably conductive polymer of the third conductor in electrical communication with the third electrode, with the third contact.

In a preferred embodiment, the method includes a step of preventing further electrical signal being passed from at least one of the conductors to the instrument configured to receive and/or supply electrical signal at a time after at least a first electrical signal has been passed from at least one of the first, second and third conductors to the instrument. In a preferred embodiment, the instrument causes an elevated current to be passed between conductors, preferably adjacent conductors, at a time after an electrical signal has been passed from at least one of the first, second and third conductors to the instrument. Preferably the elevated current is sufficient to prevent further electrical signal being passed from the conductors to the instrument i.e. at least one of the conductors may be made non-conductive. Preferably, the elevated current destroys a narrow conductor/conductive track of a test device as defined herein.

The invention also provides an instrument configured to receive and supply electrical signal that is further configured to, at a time after at least a first electrical signal has been passed from a test device (preferably a test device comprising a conductive polymer) to the instrument, prevent further electrical signal being passed to the instrument. Preferably the test device is a test device of the invention. Preferably, the instrument is configured to cause an elevated current to be passed between conductors or conductive tracks of a test strip, preferably adjacent conductors or conductive tracks. Preferably, one of the conductors or conductive tracks is a narrow conductor or conductive track as defined in relation to the first aspect of the invention. Preferably, the elevated current is sufficient to destroy the narrow track, in order to “fuse” the test device or make the narrow track non-conductive.

The invention also provides a test device comprising a narrow conductive track and a non-narrow conductive track, the narrow conductive track being configured to be made non-conductive, preferably destroyed when an elevated current is passed between the narrow conductive track and the non-narrow conductive track. Preferably, the test device is configured to receive the elevated current from an instrument configured to supply and receive electrical signal, such as a test meter.

Preferably, the test device is configured such that once the narrow conductive track has been made non-conductive, electrical signal cannot be passed to the instrument configured to supply and receive electrical signal.

The invention also provides the combination of an instrument configured to receive and supply electrical signal and a test device comprising a narrow conductive track and a non-narrow conductive track as defined herein.

The elevated current may be greater than or equal to 0.5 A, 0.6 A, 0.7 A, 0.8 A, 0.9 A, 1 A, 1.1 A, 1.2 A, 1.3 A, 1.4 A, 1.5 A, 1.6 A, 1.7 A, 1.8 A, 1.9 A or 2 A.

The first electrode and/or the first conductor, the second electrode and/or the second conductor and the third electrode and/or the third conductor may be disposed within a test device or test strip. Preferably, the test device is a test device according to the first aspect of the invention.

Thus, the engaging step may include inserting a test device (such as a test strip) comprising (i) the first conductor (ii) the first conductor and the second conductor, or (iii) the first, second and third conductors into the instrument configured to receive and/or supply the electrical signal. The method may also comprise removing the test device from the instrument.

The instrument configured to send and/or receive an electrical signal may be a test meter such as a glucose meter. The conductive polymer of any one the first, second and third conductors and/or any one of the first, second and third electrodes may be any conductive polymer defined in relation to the first aspect of the invention. The reagent may be as defined in relation to the first aspect of the invention and may include as the mediator compound, any mediator compound disclosed herein.

The method is useful for the detection of an analyte in a sample comprising a bodily fluid. The bodily fluid is preferably selected from blood, plasma, serum, cerebrospinal fluid, urine, saliva, sputum and semen. Preferably, the analyte is glucose, although it will be immediately apparent to the skilled person that the method can be adapted to detect a wide range of analytes by selecting an appropriate reagent. The analyte can be any analyte (or derivative of an analyte) for which there is a suitable oxidoreductase enzyme which can oxidise or reduce the electrode as described herein. For example, the analyte may be selected from lactic acid, alcohol, hydroxybutyrate, cholesterol, amino acids, pyruvic acid, hydrogen peroxide, sarcosine, amines, glycerol, uric acid, xanthine, ascorbic acid, NAD⁺, NADH, NAD⁺, NADPH, creatinine, lipids and ketones. When the target is glucose, the reagent preferably includes at least one enzyme configured to facilitate detection of glucose, and the instrument includes a glucose meter.

In a fourth aspect, the invention provides a method, comprising:
contacting (a) a sample comprising a bodily fluid and (b) a reagent configured to facilitate detection of an analyte in the bodily fluid with a sensor; and
either (i) monitoring a change in the colour of a conductive polymer portion of the sensor which is in electrical communication with the sample and the reagent, at least a portion of the conductive polymer being spaced apart from the sensor in contact with the bodily fluid and reagent,
or (ii) passing an electrical signal from the sensor along a conductive polymer portion of the sensor and monitoring a change in colour of a portion of the conductive polymer which is in electrical communication with the sample, at least a portion of the conductive polymer being spaced apart from the sensor in contact with the bodily fluid and reagent.

The sensor may be in electrical communication with a component of the sample. The component may or may not be the analyte. Where the component is not the analyte, it is preferably a component that can be oxidised and/or reduced and may not participate directly in the detection of an analyte, but may be provided to maintain, facilitate or support the electrical aspect of the system.

Preferably, the method facilitates detection of the analyte. The analyte, bodily fluid, reagent, conductive polymer may be as defined in relation to any other aspect of the invention.

In a fifth aspect the invention provides a compound or salt thereof, having a formula selected from:

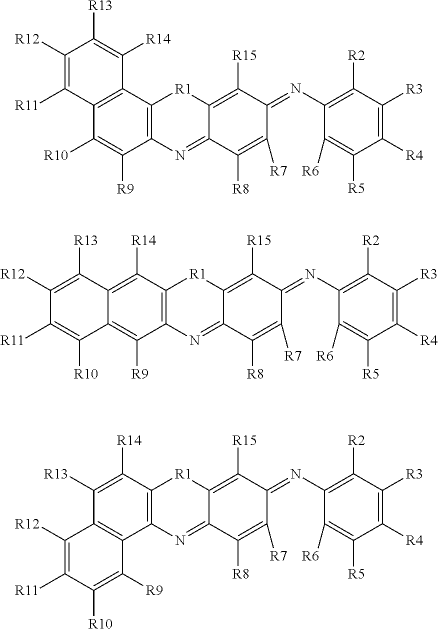

wherein R is either 0 or S and R2-R15 may be the same or different and may be independently selected from the group comprising hydrogen; sulfonyl; carboxyl; hydroxyl; C1-12 unsubstituted, substituted, linear or branched alkyl, alkenyl or alkynyl; amino; amido; aryl, halo alkoxy, nitro, and further wherein two adjacent R groups may be taken together to form an aryl, heteroaryl, cycloalkyl or cycloheteroaryl group.

In preferred embodiments, R2-R15 are each independently selected from the group comprising hydrogen, sulfonyl and carboxyl.

In a sixth aspect, the invention provides a compound or salt thereof, having a formula selected from:

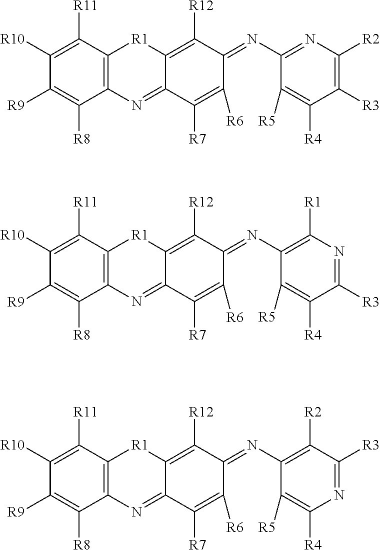

wherein R1 is either 0 or S and R2-12 may be the same or different and may be independently selected from the group comprising hydrogen; sulfonyl; carboxyl; hydroxyl; C1-12 unsubstituted, substituted, linear or branched alkyl, alkenyl or alkynyl; amino; amido; aryl, halo, alkoxy, nitro, and further wherein two adjacent R groups may be taken together to form an aryl, heteroaryl, cycloalkyl or cycloheteroaryl group.

In preferred embodiments, R2-R12 are each independently selected from the group comprising hydrogen, sulfonyl and carboxyl.

In a seventh aspect, the invention provides a compound or salt thereof, having the formula:

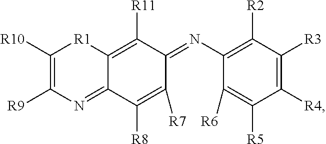

wherein R1 is either O or S and R2-R11 may be the same or different and may be independently selected from the group comprising hydrogen; sulfonyl; carboxyl; hydroxyl; C1-12 unsubstituted, substituted, linear or branched alkyl, alkenyl or alkynyl; amino; amido; aryl, halo, alkoxy, nitro, and further wherein two adjacent R groups may be taken together to form an aryl, heteroaryl, cycloalkyl or cycloheteroaryl group.

In preferred embodiments, R2-R11 are each independently selected from the group comprising hydrogen, sulfonyl and carboxyl.

In an eighth aspect, the invention provides a compound or salt thereof, having the formula:

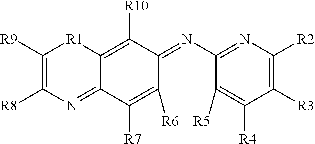

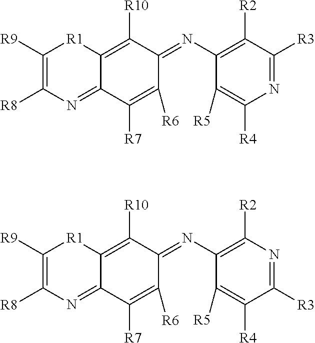

wherein R1 is either 0 or S and R2-10 may be the same or different and may be independently selected from the group comprising hydrogen; sulfonyl; carboxyl; hydroxyl; C1-12 unsubstituted, substituted, linear or branched alkyl, alkenyl or alkynyl; amino; amido; aryl, halo, alkoxy, nitro, and further wherein two adjacent R groups may be taken together to form an aryl, heteroaryl, cycloalkyl or cycloheteroaryl group.

In preferred embodiments, R2-R10 are each independently selected from the group comprising hydrogen, sulfonyl and carboxyl.

In a ninth aspect, the invention provides a compound or salt thereof, having the formula:

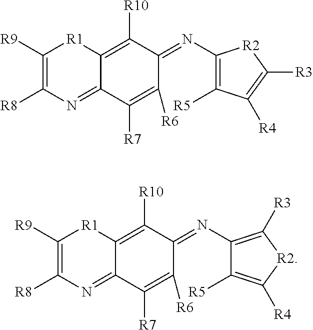

wherein R1 is either O or S, R2 is either O, S or NH and R3-10 may be the same or different and may be independently selected from the group comprising hydrogen; sulfonyl; carboxyl; hydroxyl; C1-12 unsubstituted, substituted, linear or branched alkyl, alkenyl or alkynyl; amino; amido; aryl, halo, alkoxy, nitro, and further wherein two adjacent R groups may be taken together to form an aryl, heteroaryl, cycloalkyl or cycloheteroaryl group.

In preferred embodiments, R3-R10 are each independently selected from the group comprising hydrogen, sulfonyl and carboxyl.

In a tenth aspect, the invention provides a compound or salt thereof, having the formula:

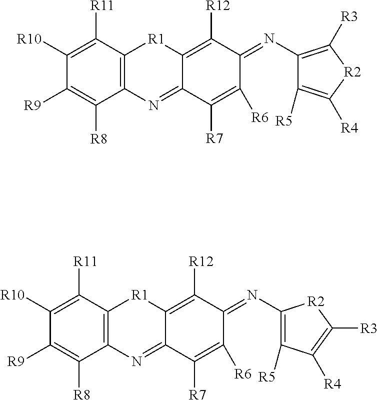

wherein R1 is either O or S, R2 is either O, S or NH and R3-12 may be the same or different and may be independently selected from the group comprising hydrogen; sulfonyl; carboxyl; hydroxyl; C1-12 unsubstituted, substituted, linear or branched alkyl, alkenyl or alkynyl; amino; amido; aryl, halo, alkoxy, nitro, and further wherein two adjacent R groups may be taken together to form an aryl, heteroaryl, cycloalkyl or cycloheteroaryl group.

In preferred embodiments, R3-R12 are each independently selected from the group comprising hydrogen, sulfonyl and carboxyl.

In an eleventh aspect, the invention provides a compound or salt thereof, having the formula:

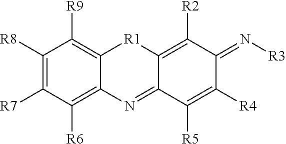

wherein R1 is S or O, R3 is either H, CH₂COOH, CH₂SO₃H, CH₂NH₂, or CH₂NO₂, and R2 and R4-R9 may be the same or different and may be independently selected from the group comprising hydrogen; sulfonyl; carboxyl; hydroxyl; C1-12 unsubstituted, substituted, linear or branched alkyl, alkenyl or alkynyl; amino; amido; aryl, halo, alkoxy, nitro, and further wherein two adjacent R groups may be taken together to form an aryl, heteroaryl, cycloalkyl or cycloheteroaryl group.

In preferred embodiments, R2 and R4-R9 are each independently selected from the group comprising hydrogen, sulfonyl and carboxyl.

An alkyl group is preferably straight or branched chain with 1 to 12 carbons. The alkyl group therefore has 1, 2, 3, 4, 5, 6, 7, 8, 9, 10, 11 or 12 carbon atoms. Specifically, examples of “C₁₋₁₂ alkyl group” include methyl group, ethyl group, n-propyl group, iso-propyl group, n-butyl group, iso-butyl group, sec-butyl group, tert-butyl group, n-pentyl group, n-hexyl group, n-heptyl group, n-octyl group, n-nonyl group, n-decyl group, n-undecyl group, n-dodecyl group, and the like.

An aryl group is a monocyclic or polycyclic ring system having from 5 to 14 carbon atoms. An aryl group is preferably a “C₆₋₁₂ aryl group” and is an aryl group constituted by 6, 7, 8, 9, 10, 11 or 12 carbon atoms and includes condensed ring groups such as monocyclic ring group, or bicyclic ring group and the like. Specifically, examples of “C₆₋₁₀ aryl group” include phenyl group, biphenyl group, indenyl group, naphthyl group or azulenyl group and the like. It should be noted that condensed rings such as indan and tetrahydro naphthalene are also included in the aryl group.

A heteroaryl group is an aryl group having, in addition to carbon atoms, from one to four ring heteroatoms which are preferably selected from O, S, N, P and Si. A heteroaryl group preferably has from 5 to 14 ring atoms. Specifically, examples of a heteroaryl group includes pyridine, imidazole, N-methylimidazole and 4-dimethylaminopyridine.

Alkenyl and alkynyl groups are preferably “C₂₋₁₂ alkenyl” and “C₂₋₁₂ alkynyl”, more preferably “C₂₋₁₀ alkenyl” and “C₂₋₁₀ alkynyl”, even more preferably “C₂₋₈ alkenyl” and “C₂₋₈ alkynyl”, most preferably “C₂₋₆ alkenyl” and “C₂₋₆ alkynyl” groups respectively.

An alkoxy group is preferably a “C₁₋₁₂ alkoxy group”, more preferably a “C₁₋₁₀ alkoxy group”, even more preferably a “C₁₋₈ alkoxy group”, even more preferably a “C₁₋₆ alkoxy group” and is an oxy group that is bonded to the previously defined C₁₋₁₂ alkyl group.

Cycloalkyl groups have from 3 to 12 carbon atoms. The cycloalkyl groups therefore have 3, 4, 5, 6, 7, 8, 9, 10, 11 or 12 carbon atoms. Specifically, examples of the C₃₋₁₂ cycloalkyl group include cyclopropyl, cyclobutyl, cyclopentyl, cyclohexyl, cycloheptyl, adamantyl and cyclooctyl.

A heterocycloalkyl group is an cycloalkyl group as defined above which has, in addition to carbon atoms, one or more ring heteroatoms, which are preferably selected from O, S, N, P and Si.

Heterocycloalkyl groups preferably contain from one to four heteroatoms, which may be the same or different.

A carboxyl group is preferably OC(O)Rₐ, wherein Rₐ can be hydrogen, an alkyl, alkenyl, alkynyl, aryl or heteroaryl group as defined above. Preferably Rₐ is hydrogen.

A sulfonyl group is a —S(O)₂OR_{b}— wherein R_{b} can be hydrogen, alkyl, alkenyl, alkynyl, aryl or heteroaryl group as defined above. Preferably R_{b} is hydrogen.

An amino group is preferably —NH₂, —NHR_{c} or —N(R_{c})₂ wherein R_{c} can be an alkyl, alkenyl, alkynyl, aryl or heteroaryl group as defined above. It will be appreciated that when the amino group is N(R_{c})₂, each R_{c} group can be the same or different. Preferably R_{c} is methyl, ethyl or propyl.

The terms “halo”, “halide” and “halogen” are used interchangeably and, as used herein mean a fluorine atom, a chlorine atom, a bromine atom, an iodine atom and the like.

A nitro group is NO₂.

Each of the above alkyl, alkenyl, alkynyl, alkoxy, aryl, heteroaryl, cycloalkyl, cycloheteroalkyl, sulfonyl, carboxyl and amino groups defined above may optional be substituted by alkyl, alkenyl, alkynyl, alkoxy, aryl, heteroaryl, cycloalkyl, cycloheteroalkyl, sulfonyl, carboxyl, amino groups halogen, nitro, cyano.

Exemplary compounds of the invention include:

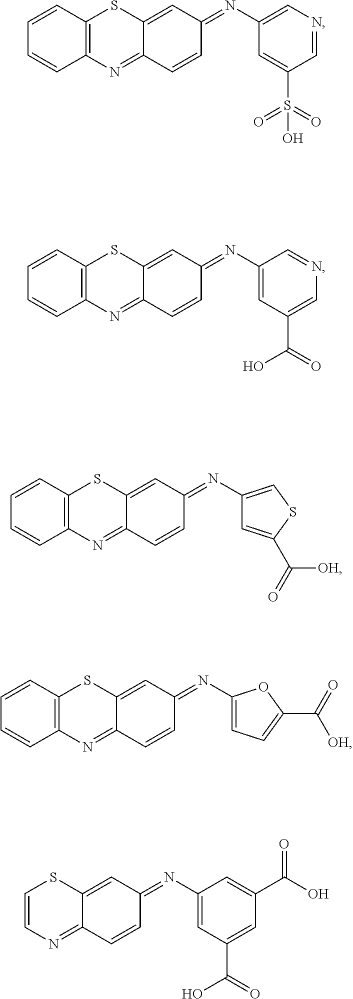 [SMILES: O=S(=O)(O)c1cncc(/N=c2\ccc3nc4ccccc4sc-3c2)c1 ; O=C(O)c1cncc(/N=c2\ccc3nc4ccccc4sc-3c2)c1 ; O=C(O)c1cc(/N=c2\ccc3nc4ccccc4sc-3c2)cs1 ; O=C(O)c1ccc(/N=c2\ccc3nc4ccccc4sc-3c2)o1 ; O=C(O)c1cc(/N=c2\ccc3nccsc-3c2)cc(C(=O)O)c1]

or salts thereof.

Any of the above compounds may be a component of a reagent composition, preferably the mediator compound, defined in relation to any of the devices and methods of the invention disclosed herein.

In a twelfth aspect, the invention provides use of one of the foregoing compounds as a component of a reagent composition for use in an electrochemical assay, wherein the reagent comprises, in addition to the compound, an oxidoreductase enzyme, and a buffer salt. Preferably, the compound is a mediator compound.

The oxidoreductase enzyme may be conjugated to an antibody and the electrochemical assay may be an electrochemical immunoassay. Preferably, the oxidoreductase enzyme converts the compound from an oxidised form to a reduced form, and wherein an electrode is used to convert the reduced compound back to the oxidised form, in so doing transferring at least one electron to the electrode which is recorded as an electrical current

In a thirteenth aspect a mixture is provided which comprises a mediator compound as defined in relation to the fifth, sixth, seventh, eighth, ninth, tenth, or eleventh aspect of the invention and a biological fluid sample, wherein the fluid is selected from blood, plasma, serum, cerebrospinal fluid, urine, saliva, sputum, semen.

Preferred features of each aspect of the invention are as for each of the other aspects mutatis mutandis.

### DESCRIPTION OF THE FIGURES

FIG. 1 shows an embodiment of a test strip of the invention.

FIG. 2 shows an exploded view of the test strip of FIG. 1.

FIG. 3 shows an embodiment of a base substrate and conductive tracks of a test strip of the invention.

FIG. 4 shows an embodiment of a test strip of the invention comprising an insulation layer disposed over a base substrate.

FIG. 5 shows an embodiment of a test strip of the invention in which a counter electrodes and a measurement electrode are provided as interdigitated fingers.

FIG. 6 shows an embodiment of a test strip of the invention comprising exposed electrode areas approximately half the area of the test strip shown in FIG. 3.

FIG. 7 shows an embodiment of a test strip of the invention comprising additional conductive tracks.

FIG. 8 shows an embodiment of a test strip of the invention comprising two narrow conductive tracks.

FIG. 9 represents a dose response profile for the amperometric measurement of glucose using several test strips according to the invention.

### DETAILED DESCRIPTION

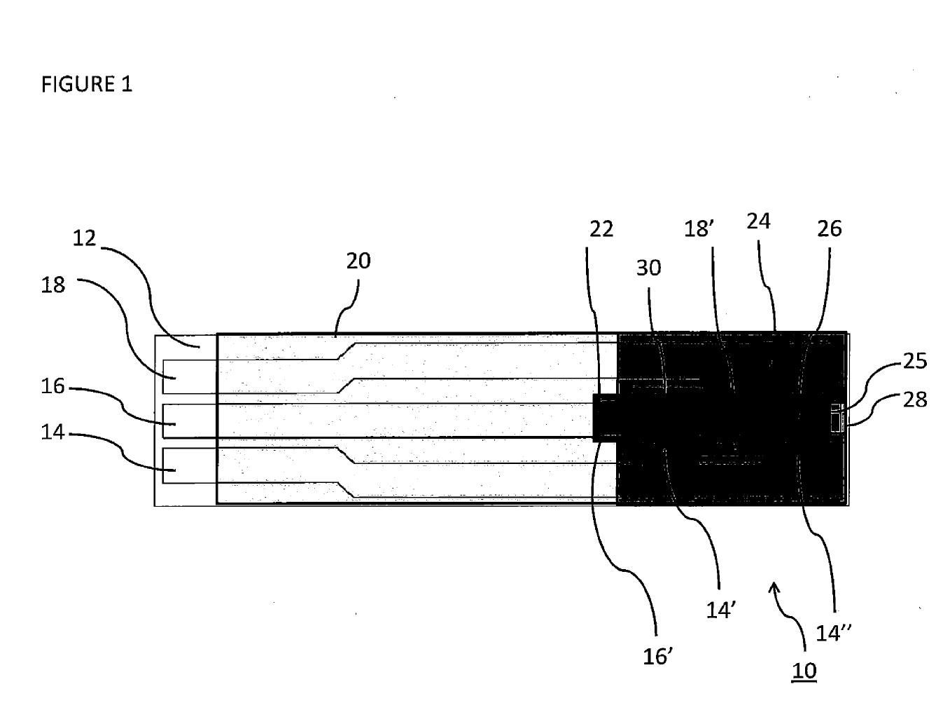

FIG. 1 shows a test strip 10 including an insulating substrate 12 on which is disposed a series of conductive tracks 14-14′,14″, 16-16′, 18-18′, over which is disposed a reagent layer 26 and an insulation layer 20. A top layer 24 is disposed over reagent layer 26 and insulation layer 20, to yield a sample chamber 30 which has a vent 22 at the opposite end of chamber 30 to a sample inlet 28. Sample chamber 30 defines a volume of between about 0.5 and 1.5 μl, and is disposed at a proximal end of test strip 10. A series of contacts 14, 16, 18 are present at a distal end of test strip 10 which engage with a connector in a meter to form an electrical connection between the meter circuitry and the test strip 10.

Conductive tracks 14-14′,14″, 16-16′, 18-18′ define respectively a counter electrode, having arms 14′ and 14″, a reference electrode 16′ and a measurement electrode 18′. Measurement electrode 18′ is positioned between arms 14′ and 14″ of the counter electrode. An insulation layer 20 is disposed over a substantial portion of the surface of insulating substrate 12 and over conductive tracks 14-14′,14″, 16-16′, 18-18′. An aperture 25 is present within insulation layer 20 which leaves exposed a portion of the conductive tracks that represent counter electrode 14′,14″, reference electrode 16′ and measurement electrode 18′. A further aperture in insulation layer 20 leaves exposed contacts 14, 16, 18 at a distal end of test strip 10.

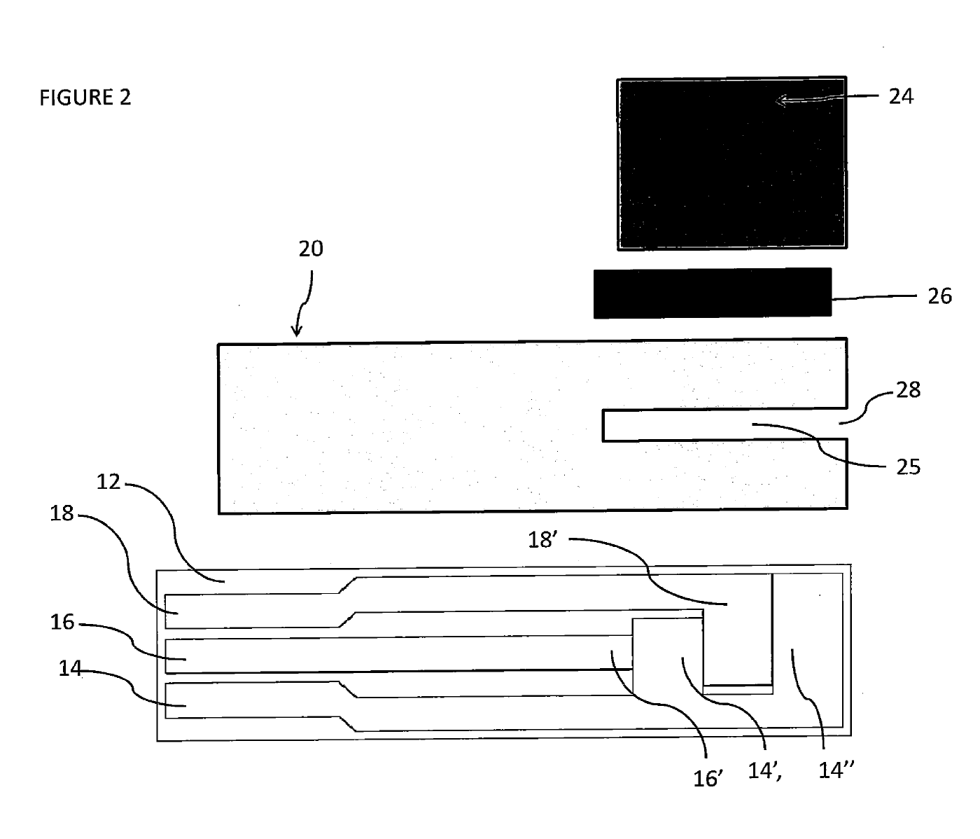

FIG. 2 shows an exploded view of the test strip 10 of FIG. 1, showing the respective layers used in construction of test strip 10, including top layer 24; reagent layer 26; insulation layer 20, comprising aperture 25 (which defines sample inlet 28); and base substrate 12, comprising conductive tracks 14-14′,14″, 16-16′, 18-18′.

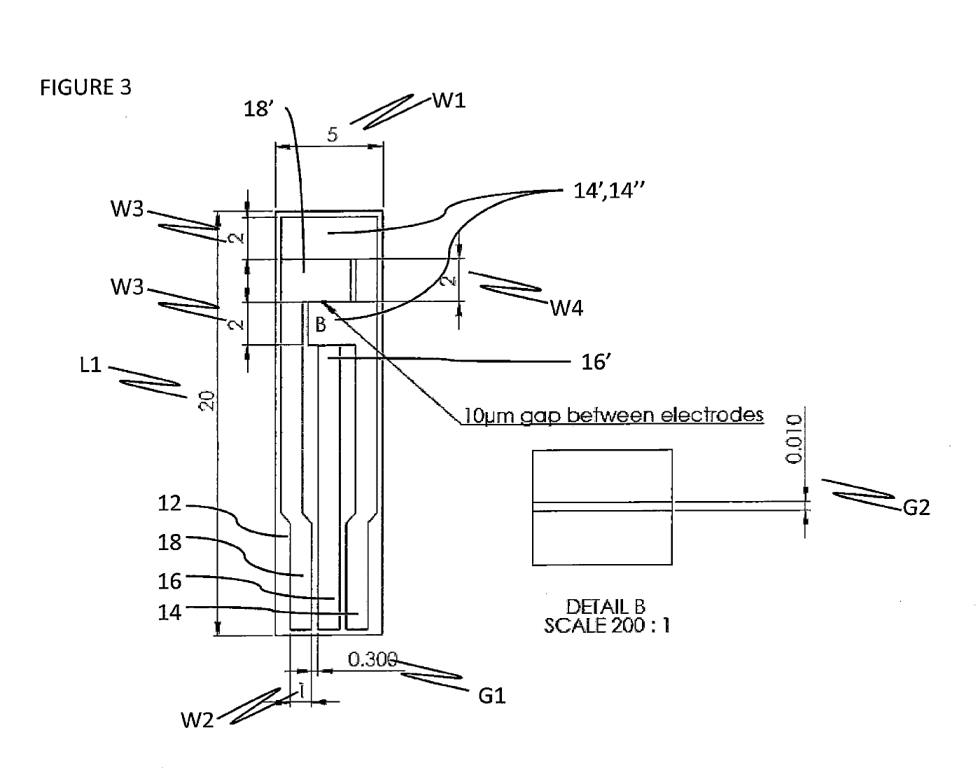

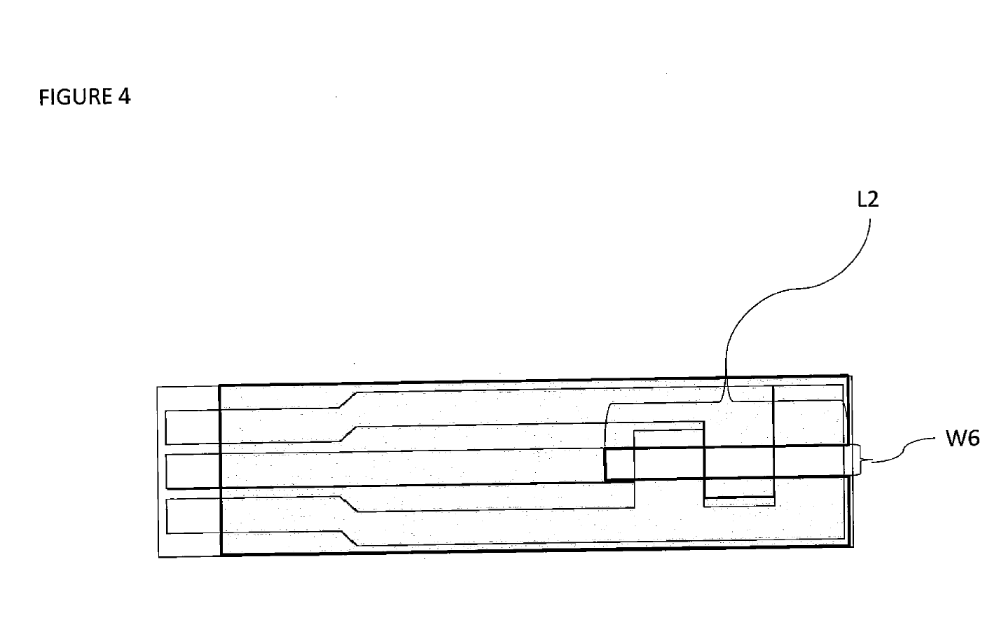

FIG. 3 shows one embodiment of base substrate 12, showing dimensions of base substrate 12 and conductive tracks 14-14′,14″, 16-16′, 18-18′. In the embodiment shown in FIG. 3, base substrate 12 has a width dimension, W1 of about 5 mm and a length dimension L1 of about 20 mm. Exposed contacts 14, 16, 18 have a width, W2 of about 1 mm, with a gap, G1, therebetween of about 0.3 mm. Counter electrodes 14′,14″ have a width W3 of about 2 mm; measurement electrode 18′ has a width W4 of about 2 mm; reference electrode 16′ has a width W2 of 1 mm. As shown in FIG. 4, when insulation layer 20 is disposed over base substrate 12, aperture 25 exposes a region having a width W6 of about 1 mm and a length L2 of about 7 mm. Electrodes 14′, 14″ and 18′ thus have exposed dimensions of about 1 mm×2 mm and electrode 16′ has an exposed area of about 1 mm×1 mm. The gap G2 (as depicted in FIG. 3) between each of electrodes 14′,14″, 16′ and 18′ is about 0.01 mm.

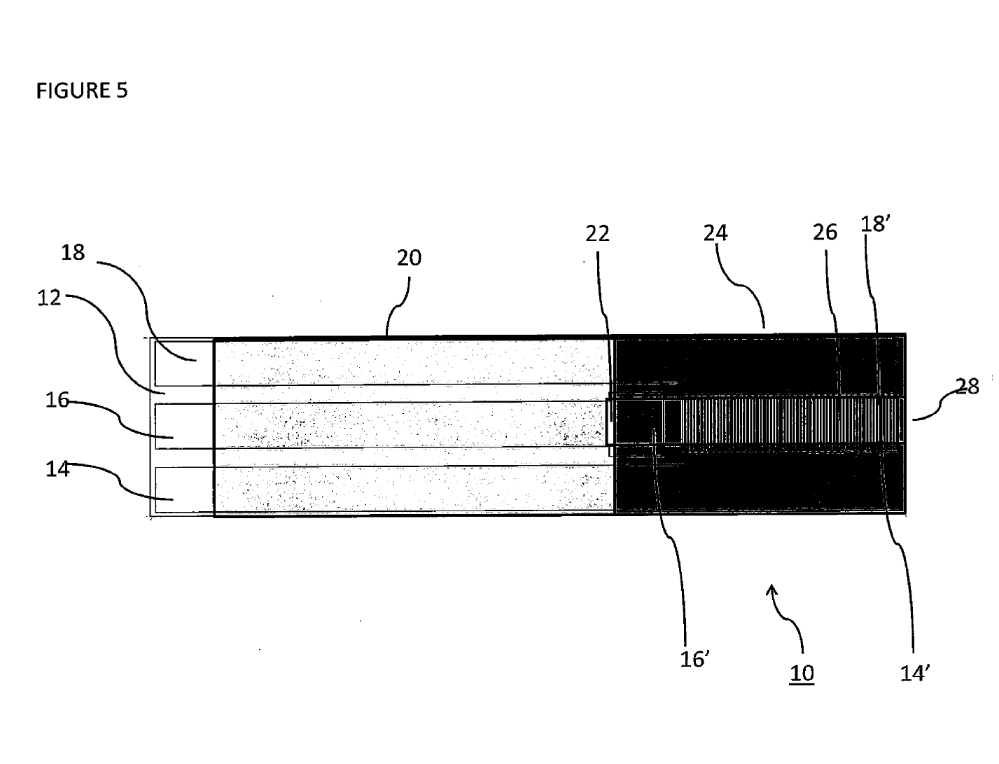

In a further embodiment, as shown in FIG. 5, counter electrode 14′ and measurement electrode 18′ are provided as interdigitated fingers, in which each respective electrode forms every other “rung” in what is presented as a “ladder” to an incoming fluid sample that is applied to sample inlet 28. When a sample of fluid is applied to sample inlet 28, the sample is drawn by capillarity into sample chamber 26. As sample is drawn into sample chamber 26, air within the chamber is vented through vent 22. When sample fluid reaches vent 22, the vent is closed and no further sample is drawn into the chamber.

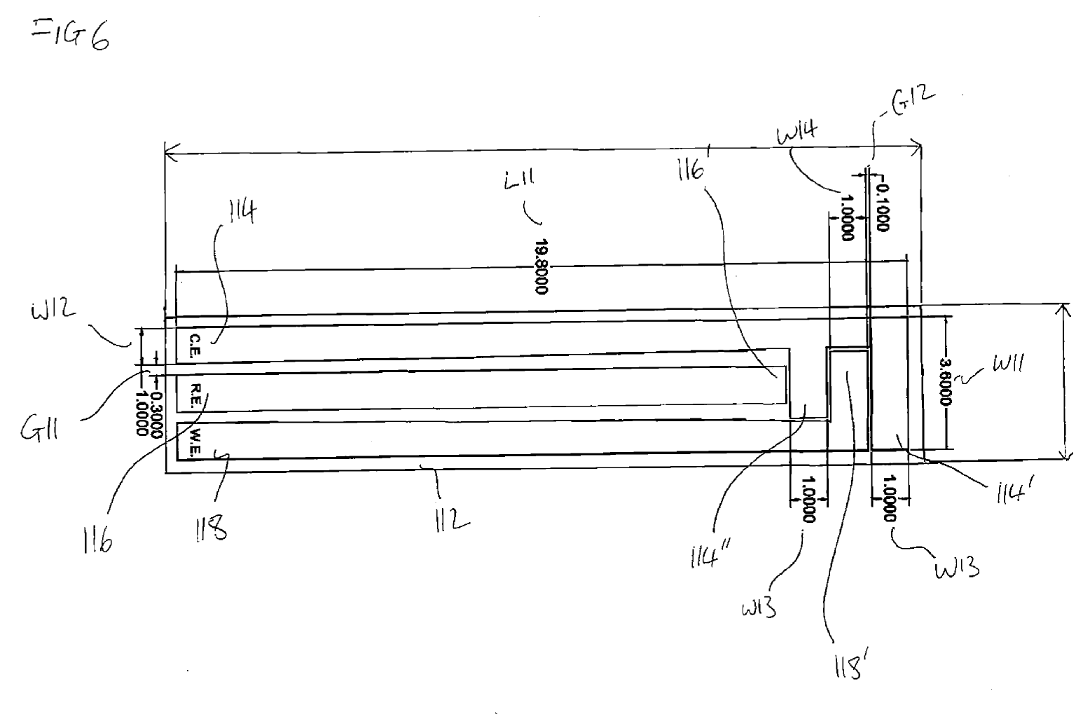

In another embodiment, as shown in FIG. 6, base substrate 112, has disposed thereon conductive tracks 114-114′,114″, 116-116′ and 118-118′ respectively. In the embodiment of FIG. 6, base substrate 12 has a width dimension, W11 of about 4 mm and a length dimension L11 of about 20 mm. Exposed contacts 114, 116, 118 have a width, W2 of about 1 mm, with a gap, G11, therebetween of about 0.3 mm. Counter electrodes 114′,114″ have a width W3 of about 1 mm; measurement electrode 118′ has a width W14 of about 1 mm; reference electrode 116′ has a width W12 of 1 mm. As shown with respect to FIG. 4, when an insulation layer 20 is disposed over the base substrate 12, an aperture 25 exposes a region having a width W6 of about 1 mm and a length L2 of about 7 mm. In the embodiment of FIG. 6, the portions of electrodes 114′, 114″ and 118′ that are exposed through aperture 25 have dimensions of about 1 mm×1 mm and electrode 116′ also has an exposed area of about 1 mm×1 mm. The gap G12 (as depicted in FIG. 6) between each of electrodes 114′,114″, 116′ and 118′ is about 0.1 mm.

The device according to FIG. 6 thus has effective exposed electrode areas approximately half the area of the device according to FIG. 3.

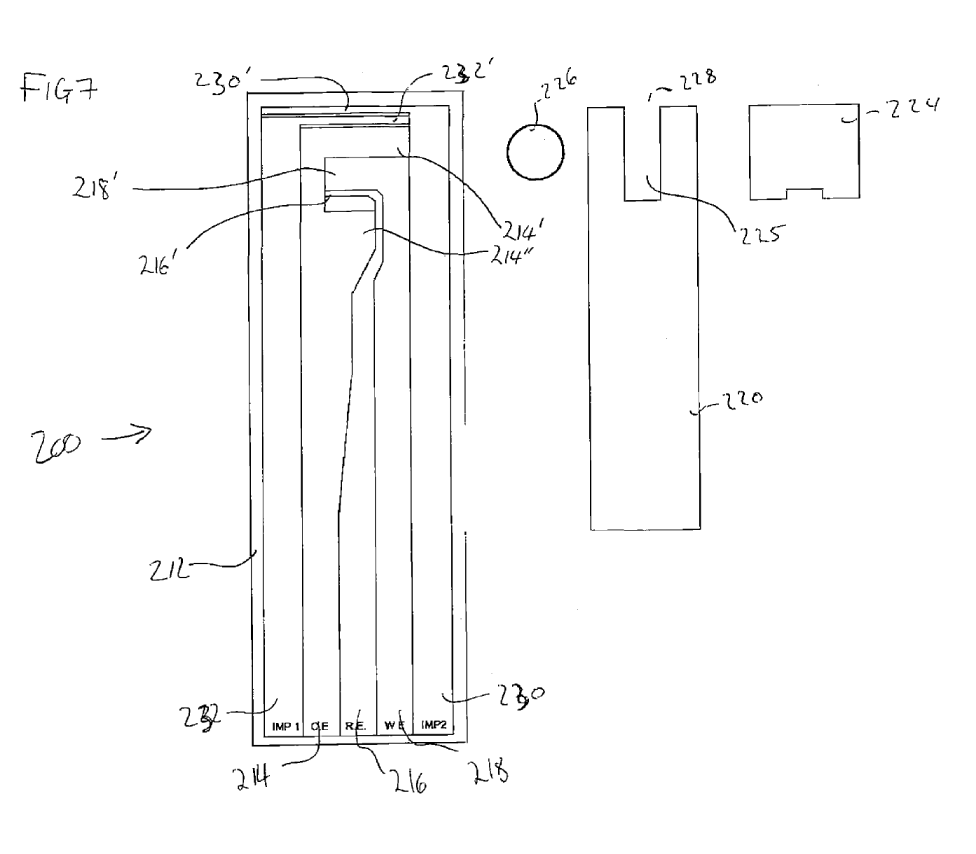

In yet a further embodiment, as depicted in FIG. 7, a base substrate 212 has disposed thereon conductive tracks 214-214′,214″, 216-216′, 218-218′, 230-230′ and 232-232′ respectively. The embodiment of FIG. 7 differs from the embodiments of FIGS. 3 and 6 in that two further conductive tracks, 230-230′ and 232-232′, are provided. Electrodes 230′ and 232′ are used to determine an impedance parameter of a sample fluid that is applied to the test strip. Changes in the properties of any given sample may give rise to a difference in the measured parameter of that sample, these properties may also give rise to a change in an impedance parameter of that sample. The inclusion of electrodes 230′ and 232′ thus provides for measurement of an impedance parameter of the sample. A correction factor as may be appropriate to compensate for variability due to sample matrix effects, as determined by a change in sample impedance may therefore be determined and applied. For example, the haematocrit of a sample of blood may have an impact on the measurement of a soluble species, such as glucose, present in the blood. Haematocrit may be determined by determining an impedance parameter of blood, as is well documented in the literature. A correction factor may subsequently be applied to compensate for any impact due to haematocrit when determining a value for blood glucose, for example.

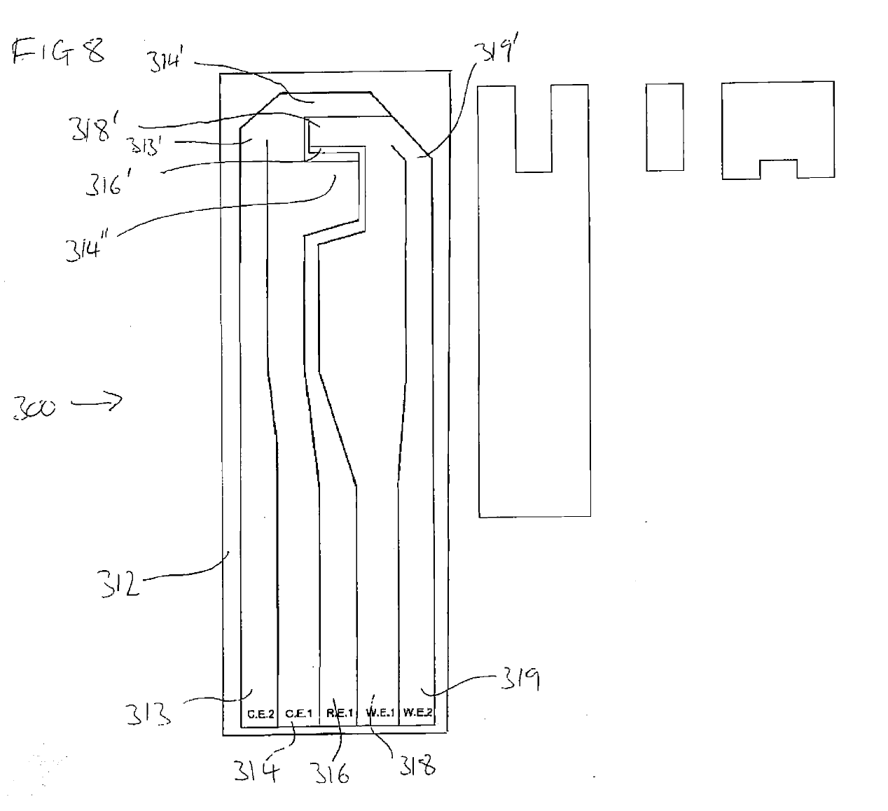

In yet another embodiment, as shown in FIG. 8, test strip 300 includes the features of the embodiment shown in FIGS. 1 to 4, with an additional narrow conductive track 319′, that connects contact 319 to measurement electrode 318′. Narrow conductive track 319′ provides a signal to a microprocessor (not shown) as part of the circuits of the meter in to which test strip 300 is inserted. By monitoring changes in the signal received via narrow conductive track 319′, the microprocessor can determine the voltage present at measurement electrode 318′. Variation in the conductivity of the various tracks that join measurement electrode 318′, counter electrode 314′, 314″ and reference electrode 316′, may result in the voltage at the measurement electrode 318′ being slightly different than would otherwise be expected based on the excitation voltage generated by the microprocessor, typically as a result of IR (current/resistance) drops along the length of the conductive track. Voltage drop or IR drop can occur along a resistive track when current flows which, in the case of a device such as that described here with reference to FIGS. 1-8, could make the voltage at the electrode portion exposed (e.g. with reference to FIG. 8, electrode 318′) to the sample different from the voltage applied at the connector in the meter (e.g. with reference to FIG. 8, contact 318). Variable resistance or length of the track could lead to variable voltage drop and variable voltage at the electrode portion exposed to the sample. According to the embodiment of FIG. 8, the width of the conductive track which runs the length of the strip from measurement electrode 318′ to contact 318 is maximised to reduce the resistance and therefore the potential IR drop in this track. The resistance of the other tracks is less critical in a three electrode system under potentiostatic control and so these tracks may be made thinner, particularly the reference electrode track which does not carry significant current and therefore does not experience significant IR drop. Voltage drop in the counter electrode track, which also carries current, is less critical than in the measurement electrode track as the potentiostat within the meter will compensate by increasing the applied voltage at the meter connector although only up to its maximum possible applied voltage. Nonetheless, the device of FIG. 8 also depicts an optional feature, which includes a further contact 313 that terminates at narrow conductive track 313′. Narrow conductive track 313′ operates similarly to narrow conductive track 319′. However, in this instance, narrow conductive track 313′ permits the microprocessor to determine the voltage present at counter electrode 314′, 314″. The inclusion of a correction amplifier circuit within the microprocessor that receives the “sensed voltage” at measurement electrode 318′ via narrow conductive track 319′ allows for greater control over the actual voltage at measurement electrode 318′. Similarly, the optional inclusion of a correction amplifier circuit within the microprocessor that receives the “sensed voltage” at counter electrode 314′, 314″ via narrow conductive track 313′ allows for greater control over the actual voltage at counter electrode 314′, 314″. Through improved closed loop feedback control, the microprocessor is better able to adjust the applied potential to maintain the desired or expected voltage at the measurement electrode 318′ (and optionally also the counter electrode 314′, 314″) in order to achieve the specific measurement in question. Greater control of the applied voltage will lead to measurement results that have improved reproducibility sample to sample, a factor that is desirable when seeking to achieve very precise measurements, particularly when the target analyte is present at low concentration, and thus where signal noise may otherwise adversely influence the response. Since neither of the tracks that connect contacts 313 or 319 to narrow conductive track 313′ or 319′ respectively carry any significant current, unlike the tracks connecting contacts 314 and 318 to electrodes 314′, 314″ and 318′ respectively, they are not affected by IR drop.

In yet a further embodiment, as will be described with reference to FIG. 8, the meter into which the test strip is inserted (not shown) prior to making a measurement of a target analyte, typically performs a number of “on board” functional diagnostic tests. Such tests are typically designed to verify the proper function of the microprocessor and circuits of the meter. One other diagnostic test often performed is to assess whether a test strip has previously been used. This might be achieved by measuring the level of current that flows through the test strip prior to sample application. Residues from a dried blood sample within a test strip could result in a higher current than would be achieved with an “unused” test strip, and thus this can serve as an indicator that a strip has been used. However, such an approach is not always reliable, and in some circumstances a user might be instructed to insert a “new” strip, even though the strip within the meter is unused and fully functional.

Thus, according to an embodiment where a strip having features as depicted in FIG. 8, in particular contact 313 or 319 and narrow conductive track 313′ or 319′ respectively is used, then following completion of a sample measurement and reporting of an analyte value to a user, the microprocessor of the meter causes an elevated voltage to be passed between contact 313 and 314, or between 318 and 319. The consequence of applying such an elevated current is to effectively “destroy” narrow conductive track 313′ or 319′, much in the way that a fuse wire is destroyed when the current flowing through it exceeds the rated threshold. In this instance, when a used strip is inserted into a meter following destruction of narrow conductive track 313′ or 319′, then no current would pass between contacts 313 and 314, or 318 and 319. As a result, there would be little or no uncertainty that a used strip had been inserted, since an unused strip would freely allow current to flow between contacts 313 and 314 or 318 and 319 respectively.

The devices described with reference to FIGS. 1-8 are typically prepared using a conducting polymer material that is applied over an insulating base layer. The conductive tracks and electrodes as have been described with respect to FIGS. 1-8 may be formed using a variety of techniques. In one embodiment a conductive material may be deposited onto a base substrate by a process of printing, such as for example screen printing, gravure printing, inkjet printing. In another embodiment, conductive material may be deposited onto the surface of base substrate by a process of slot die coating, vapour phase deposition, spin coating, k-bar coating, or the like, which forms a layer of uniform thickness across the entire surface of base substrate. A process of laser ablation may subsequently be used to remove specific portions of the conductive material to reveal discrete and electrically isolated conductive tracks (e.g. for example elements 14-14′,14″, 16-16′, 18-18′ as described with reference to FIG. 1). In a specific embodiment the conductive polymer is a composition comprising poly(3,4-ethylenedioxythiophene):polystyrene sulphonate (PEDOT:PSS). PEDOT:PSS is commercially available from a number of suppliers, including AGFA Gevaert BV (Mortsel, Belgium) which supplies material under the tradename Orgacon™, which include for example ELP-3145, ELP-5015, S-305+; Heraeus Precious Metals (Leverkusen, Germany), which supplies material under the tradename Clevios™, which include for example PH 1000, S V3, S V4, P Jet N V2; TDA Research, Inc. (Colorado, USA), which supplies materials under the tradename Oligotron™. PEDOT:PSS is typically supplied as a formulation containing 1-2% solids by weight of the PEDOT:PSS polymer, which is dispersed in a solvent matrix, which may be organic or inorganic, that can contain a range of additional binders and additives (including other solids) that improve adhesion of the material to a substrate surface and which can alter the conductivity of the dried polymer layer depending on the specified purpose.

In an exemplary embodiment, a PEDOT:PSS composition may comprise between about 5% to 10% by volume diethylene glycol; between about 60% to 80% by volume propylene glycol; and between about 1.5% to 5.5% weight per volume solids. The formulation may have a viscosity of between about 10 cP to about 30 cP (at 20° C.) and a dry film surface resistivity of between about 50 ohm/square to about 500 ohm/square.

With reference to FIG. 1, a film of PEDOT:PSS (such as for example Orgacon™ ELP-3145; Orgacon™ S-305+, Clevios™ SV 4) is first deposited onto a base substrate, which is typically an insulating substrate such a polyester, or polystyrene. A wet film thickness of a PEDOT:PSS preparation of at least about 51-m, at least about 7 μm, at least about 10 μm, at least about 12 μm, at least about 15 μm, at least about 17 μm, at least about 20 μm, at least about 22 μm, at least about 24 μm, at least about 26 μm, at least about 28 μm, at least about 32 μm, at least about 36 μm, at least about 40 μm is deposited over the base substrate. The wet film is subsequently dried by passage through a drying over, which may be a forced air dryer or an infra-red dryer, at a temperature of at least about 80° C., at least about 90° C., at least about 100° C., at least about 110° C., at least about 120° C., at least about 130° C., at least about 140° C., or at least about 150° C. to yield a dry film of PEDOT:PSS.

Thereafter a layer of insulating material (insulation layer 20) is applied over the dried PEDOT:PSS layer. The insulation layer serves to expose defined regions of the PEDOT:PSS layer into which a liquid sample may come in contact. The insulation layer thereby defines the surface area of the respective electrodes (14′, 14″, 16′ and 18′) that are exposed to sample and which therefore take part in a sample measurement process. The insulating material may be a screen printed dielectric ink, such as for example 118-08 from Creative Materials, Inc. Alternatively, the insulating material may be a double sided adhesive tape which has a pre-cut aperture to define the region of each electrode that would be exposed to liquid sample.

Following application of the insulation layer, a reagent layer is applied. Following this, a cover layer is placed over the dried reagent to create an enclosed cavity having a defined volume, such that when a liquid sample is applied to the device, the dried reagent is re-suspended into the defined volume of liquid applied, thereby resulting in a defined concentration of reagent within the liquid sample.

### Example

A series of reagent compositions were prepared using 16 Units of glucose dehydrogenase FAD, 25 mM mediator compound, 200 mM buffer salt (MOPS (hemisodium 3-(N-morpholino) propanesulfonate)), and 0.2% v/v surfactant (Tween® 20) and additives (1% w/v Na₂SO₄, 1 mM; hexammineruthenium (Ill) chloride). Each reagent composition was used to manufacture several test strips, each of which was used to evaluate the performance of the respective mediator within the reagent composition when glucose containing blood samples were applied to devices. A quantity of venous blood was obtained from a healthy volunteer, the blood was rolled overnight on rotating rocker such that depletion of any endogenous glucose occurs due to cellular metabolism of the sample, as will be understood by the skilled person. The blood sample, depleted of endogenous glucose, was divided into 7 aliquots, to which were added glucose to yield a notional concentration of about 0, 100, 200, 350, 420 and 600 mg/dL glucose respectively. Each blood sample was tested in replicates of five on the various test strips that were produced containing reagent formulations including different mediator concentrations.

The data obtained indicate there to be different responses to glucose according to the mediator compound present in the reagent composition. Both the gradient and intercept on the y-axis differ according to the mediator compound used. Although there were difference in slope, between each mediator compound evaluated, all compounds were shown to result in a composition that could be used to evaluate to amount of glucose present in each of the samples tested. A steeper gradient will typically allow for greater discrimination between concentrations of glucose, especially at lower concentration levels, since there is a greater difference in measured response per unit concentration along the x-axis. However, a shallower gradient might be more useful when measuring particularly high concentrations, which might otherwise result in a flattening off of the response profile at elevated glucose concentration. Thus according to the intended purpose of the particular reagent composition, a particular mediator compound may be selected to achieve the desired gradient value of the dose response profile.

With respect to the intercept on the y-axis, generally the higher the value, the higher the minimum detection limit becomes, however, this depends on the precision of the measurement at low or zero sample concentration. It might be expected that in the presence of zero target substance, the assay should report zero response; however this is rarely the case due to a variety of reasons, including non-specific interactions, components of the sample interacting at the sensor surface giving rise to low level signals. Thus the intercept on the y-axis effectively dictates the lowest measurable quantity of target sample that can be achieved under a specific set of experimental conditions. For those instances where devices are required to measure very low levels of target analyte, it is thus desirable to have a configuration that displays a low intercept couple with a steep gradient, such that there is maximum difference between measured values for points along the x-axis, particularly where those points along the x-axis are close to zero.

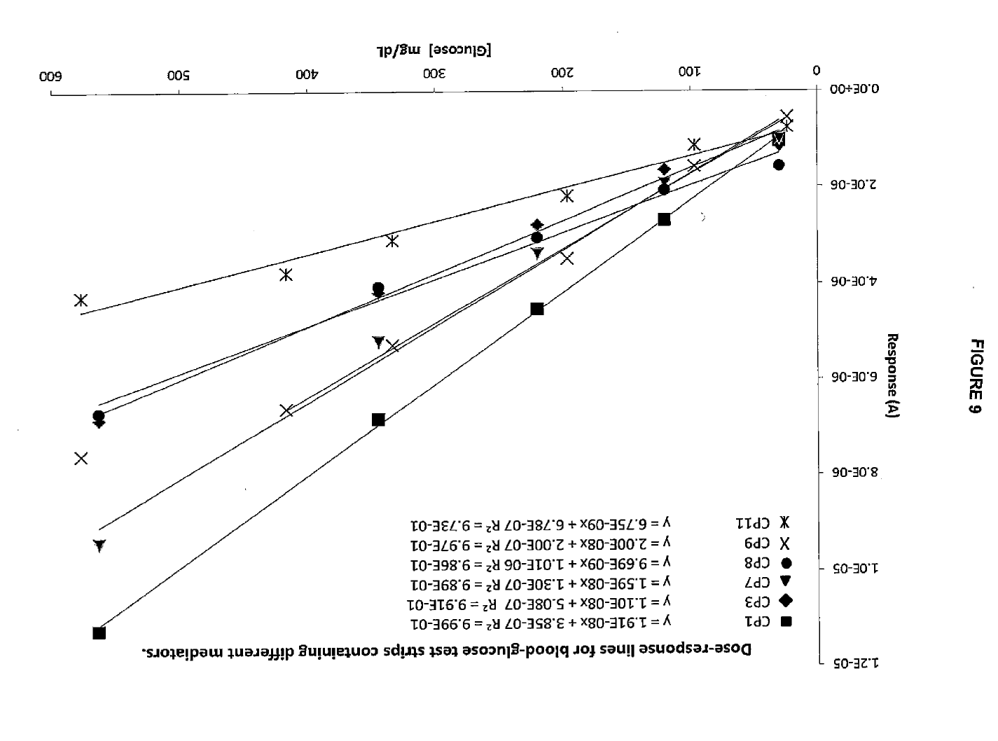

As can be seen from FIG. 9, the compound designated CP1 demonstrates a gradient (1.91E-8 mg/dL/A), while the compound designated CP9 had a gradient of (1.54E-8 mg/dl/A). The intercept value for CP1 is almost double that for CP9 (3.85E-7 A vs 1.91E-7 A respectively). The data shown in FIG. 9 might thus suggest that CP9 would result in a test strip that achieves good discrimination between samples at lower concentrations of glucose, while also displaying good separation between samples across the concentration range studied.

### Key to FIG. 9:

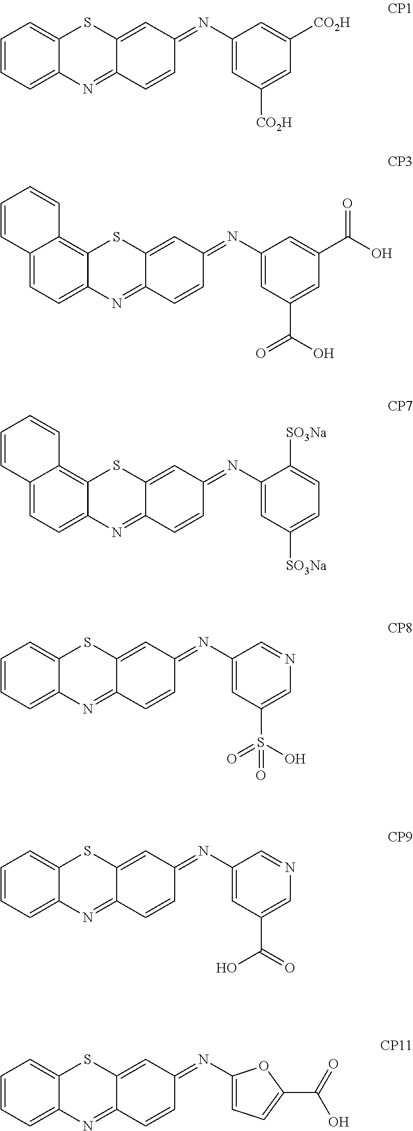

1. A test device comprising;
a substrate having disposed thereon two or more conductive tracks;
a reagent composition disposed over a portion of at least one conductive track; and
a top layer covering a portion of the two or more conductive tracks which forms in combination with the substrate a sample receiving chamber;
wherein at least one conductive track comprises a conductive polymer.

2. The test device of claim 1, wherein the device comprises two, three, four, five or six conductive tracks and each track comprises a conductive polymer.

3. The device of claim 1 or claim 2, wherein the conductive polymer comprises polythiophene, polypyrrole, polyaniline, polyfluorene, polyacetylene, poly(p-phenylene vinylene), poly(3,4-ethylenedioxythiophene), poly(3,4-propylenedioxythiophene), poly(3,3-dibenzyl-3,4-propylenedioxythiophene), poly(3-4-ethylenedioxythiophene), bis-poly(ethyleneglycol), lauryl terminated, poly(3,4-ethylenedioxythiophene)-block PEG, and poly(3,4-ethylenedioxythiophene), tetramethacrylate end-capped or combinations thereof.

4. The device of any preceding claim, wherein the conductive polymer is a complex comprising poly(3,4-ethylenedioxythiophene) and a counterion.

5. The device of claim, wherein the counterion is selected from polystyrene sulfonate (PSS), perchlorate, perchlorate p-toluene, sulfonate p-toluene, tosylate.

6. The device of claim 5, wherein the conductive polymer is a complex comprising poly(3,4-ethylenedioxythiophene) and polystyrene sulfonate (PEDOT:PSS).

7. The device of any preceding claim, wherein the at least one conductive track comprising a conductive polymer further comprises another conductive material selected from the group comprising carbon, gold, platinum, silver, palladium, copper, indium tin oxide and combinations thereof.

8. The device of any preceding claim, wherein the reagent composition comprises an oxidoreductase enzyme and a mediator compound.

9. The device of claim 8, wherein the oxidoreductase is selected from the group consisting of glucose oxidase, glucose dehydrogenase, lactate dehydrogenase, alcohol dehydrogenase, hydroxybutyrate dehydrogenase, cholesterol oxidase, amino acid oxidase, pyruvate oxidase, peroxidase, sarcosine oxidase, lactate oxidase, alcohol oxidase, monoamine oxidase, glycerol oxidase, glycerol phosphate oxidase, urate oxidase, xanthine oxidase, ascorbate oxidase, catalase, diaphorase and combinations thereof.

10. The device of claim 9, wherein the glucose dehydrogense is selected from a quinoprotein glucose dehydrogenase, FAD dependent glucose dehydrogenase, and NAD dependent glucose dehydrogenase.

11. The device of claim 10, wherein the FAD dependent glucose dehydrogenase is FAD dependent Glucose Dehydrogenase from Sekisui Diagnostics, Catalogue Number GLDE-70-1192 (E.C. number 1.1.99.10) from Aspergillus sp. or FAD dependent Glucose Dehydrogenase from BBI enzymes, Catalogue Number GLD1.

12. The device of any one of claims 8-11, wherein the mediator compound is selected from the group comprising potassium ferricyanide, ferrocene derivatives, phenoxazine derivatives, phenothiazine derivatives, quinone derivatives, and reversible redox transition metal complexes, particularly those of Ruthenium and Osmium, nicotinamide adenine dinucleotide (phosphate), diimines, phenanthroline derivatives, dichlorophenolindophenol, tetrazolium dyes, phenylimino-benzophenoxazine and combinations thereof.

13. The device of any one of claims 8-12, wherein the mediator compound is 3-(3′,5′-dicarboxy-phenylimino)-3H-phenothiazine.

14. The device of any one of claims 8-13, wherein the oxidoreductase enzyme and/or the mediator compound is incorporated within or attached to the conducting polymer by way of chemical bonding or physical entrapment.

15. The device of claim 14, wherein the mediator is attached to the conducting polymer using a linker molecule that provides for migration of the mediator between an active site of the oxidoreductase enzyme molecule and an electrode surface, thereby facilitating transfer of electrons from the enzyme to the electrode.

16. A method of manufacturing a test strip, comprising;
forming a layer of conductive polymer;
defining at least two electrically insulated conductive tracks, wherein at least one of the tracks comprises a conductive polymer;
applying a reagent composition over a portion of a portion of at least one of the tracks; and
forming a sample receiving chamber over the reagent composition, and a portion of at least one of the tracks.

17. The method of claim 16, further comprising forming a layer of conductive material other than conductive polymer.

18. The method of claim 16 or 17, wherein the step of forming a layer of conductive polymer and/or, where present, conductive material, comprises applying said material to an insulating substrate.

19. The method of any one of claims claim 16-18, wherein the step of defining the electrically insulated conductive tracks is concurrent with the step of forming a layer of conductive polymer and, where present, conductive material.

20. The method of any one of claims 16-19, wherein screen printing, gravure printing, ink-jet printing is used to define the at least two electrically insulated conductive tracks.

21. The method of claim 16 or 17, wherein the conductive polymer and/or conductive material, where present is coated on the insulating substrate and patterned by a process of laser ablation to define the at least two electrically insulated conductive tracks.

22. The method of any one of claims 16-21, wherein an insulating layer is applied over at least a portion of the conductive polymer and, where present, conductive material, to define an area that is for exposure to a sample.

23. A method, comprising:
contacting (a) a sample comprising a bodily fluid and (b) a reagent configured to facilitate detection of an analyte in the bodily fluid with a first electrode; and
passing an electrical signal from the first electrode along a first conductor in electrical communication with the first electrode, at least a portion of the first conductor being spaced apart from the first electrode in contact with the bodily fluid and reagent, wherein the first conductor comprises a conductive polymer.

24. The method of claim 23, wherein:
the step of contacting further comprises contacting (a) the sample comprising the bodily fluid and (b) the reagent configured to facilitate detection of an analyte in the bodily fluid with a second electrode, the second electrode being spaced apart from the first electrode; and passing a second electrical signal along a second conductor, the second conductor being in electrical communication with the second electrode and spaced apart from the first conductor and the first electrode.

25. The method of claim 24, wherein the second conductor comprises or consists of a conductive polymer in electrical communication with the second electrode.

26. The method of claim 24 or claim 25 wherein:
the step of contacting further comprises contacting (a) the sample comprising the bodily fluid and (b) the reagent configured to facilitate detection of an analyte in the bodily fluid with third electrode, the third electrode being spaced apart from the first and second electrodes; and passing a third electrical signal along a third conductor, the third conductor being in electrical communication with the third electrode and spaced apart from the first and second conductors and the first and second electrodes.

27. The method of claim 26, wherein the third conductor comprises or consists of a conductive polymer in electrical communication with the third electrode.

28. The method of any of claims 23-27, further comprising passing the electrical signal passed along the first conductor to a contact of an instrument configured to receive and/or supply the electrical signal.

29. The method of claim 28, further comprising mechanically engaging the first conductor with the contact.

30. The method of any of claims 24-29, further comprising (a) passing the electrical signal passed along the first conductor to a first contact of an instrument configured to receive and/or supply the electrical signal and (b) passing the electrical signal passed along the second conductor to a second contact of the instrument configured to receive and/or supply the electrical signal.

31. The method of claim 30, further comprising mechanically engaging the first conductor with the first contact and engaging the second conductor with the second contact.

32. The method of claim 31, wherein engaging the second conductor with the second contact comprises mechanically engaging the second conductor with the second contact.

33. The method of any one of claims 26 to 32, further comprising (a) passing the electrical signal passed along the first conductor to a first contact of an instrument configured to receive the electrical signal, (b) passing the electrical signal passed along the second conductor to a second contact of the instrument configured to receive and/or supply the electrical signal, and (c) passing the electrical signal passed along the third conductor to a third contact of the instrument configured to receive and/or supply the electrical signal.

34. The method of claim 33, further comprising mechanically engaging the first conductor with the first contact, engaging the second conductor with the second contact, and engaging the third conductor with the third contact.

35. The method of claim 34, wherein engaging the second conductor with the second contact comprises mechanically engaging the conductive polymer of the second conductor with the second contact and engaging the third conductor with the third contact comprises mechanically engaging conductive polymer of the third conductor with the third contact.

36. The method of any one of claims 29-35, wherein the engaging comprises inserting (i) a test strip comprising the first conductor, (ii) a test strip comprising the first and second conductors, or (iii) a test strip comprising the first, second and third conductors into the instrument.

37. The method of claim 36, wherein the test strip is a disposable test strip and the method comprises removing the test strip from the instrument.

38. The method of any one of claims 28-37, wherein the instrument configured to supply and/or receive the electrical signal causes an elevated current to be passed between adjacent conductors at a time after electrical signal has been passed from at least one of the first, second and third conductors to the instrument, such that at least one of the conductors is prevented from passing any further electrical signal to the instrument.

39. The method of any one of claims 23-38, wherein the target is glucose, the reagent comprises at least one enzyme configured to facilitate detection of glucose, and the instrument comprises a glucose meter.

40. The method of any one of claims 23-39, wherein the bodily fluid is blood.

41. A method, comprising:
contacting (a) a sample comprising a bodily fluid and (b) a reagent configured to facilitate detection of an analyte in the bodily fluid with a sensor; and
either (i) monitoring a change in the colour of a conductive polymer portion of the sensor which is in electrical communication with the sample and the reagent, at least a portion of the conductive polymer being spaced apart from the sensor in contact with the bodily fluid and reagent,
or (ii) passing an electrical signal from the sensor along a conductive polymer portion of the sensor and monitoring a change in colour of a portion of the conductive polymer which is in electrical communication with the sample, at least a portion of the conductive polymer being spaced apart from the sensor in contact with the bodily fluid and reagent.

42. A compound or salt thereof having a formula selected from: 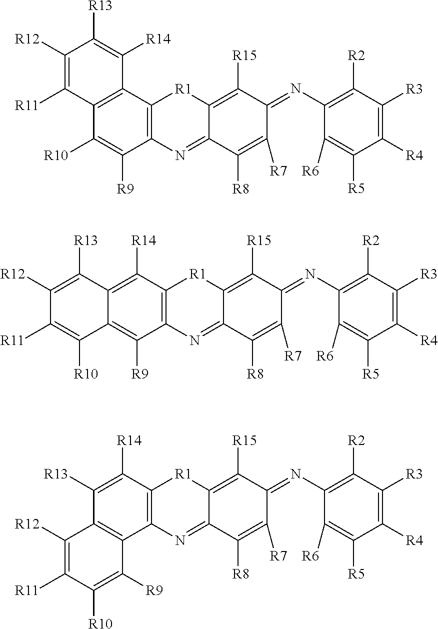
wherein R1 is either O or S and R2-R15 may be the same or different and may be independently selected from the group comprising hydrogen; sulfonyl; carboxyl; hydroxyl; C1-12 unsubstituted, substituted, linear or branched alkyl, alkenyl or alkynyl; amino; amido; aryl, halo, alkoxy, nitro, and further wherein two adjacent R groups may be taken together to form an aryl, heteroaryl, cycloalkyl or cycloheteroaryl group.

43. A compound or salt thereof having a formula selected from: 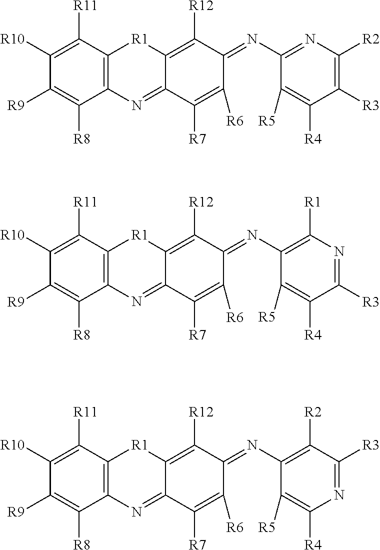
wherein R1 is either O or S and R2-12 may be the same or different and may be independently selected from the group comprising hydrogen; sulfonyl; carboxyl; hydroxyl; C1-12 unsubstituted, substituted, linear or branched alkyl, alkenyl or alkynyl; amino; amido; aryl, halo, alkoxy, nitro, and further wherein two adjacent R groups may be taken together to form an aryl, heteroaryl, cycloalkyl or cycloheteroaryl group.

44. A compound or salt thereof having the formula: 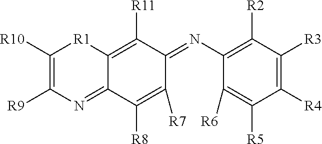
wherein R1 is either O or S and R2-R11 may be the same or different and may be independently selected from the group comprising hydrogen; sulfonyl; carboxyl; hydroxyl; C1-12 unsubstituted, substituted, linear or branched alkyl, alkenyl or alkynyl; amino; amido; aryl, halo, alkoxy, nitro, and further wherein two adjacent R groups may be taken together to form an aryl, heteroaryl, cycloalkyl or cycloheteroaryl group.

45. A compound or salt thereof having a formula selected from: 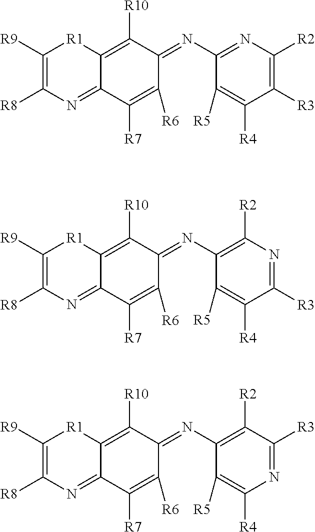
wherein R1 is either O or S and R2-10 may be the same or different and may be independently selected from the group comprising hydrogen; sulfonyl; carboxyl; hydroxyl; C1-12 unsubstituted, substituted, linear or branched alkyl, alkenyl or alkynyl; amino; amido; aryl, halo, alkoxy, nitro, and further wherein two adjacent R groups may be taken together to form an aryl, heteroaryl, cycloalkyl or cycloheteroaryl group.

46. A compound or salt thereof having a formula selected from: 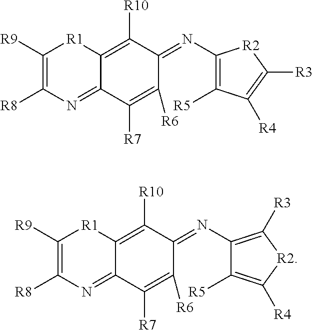
wherein R1 is either O or S, R2 is either O, S or NH and R3-10 may be the same or different and may be independently selected from the group comprising hydrogen; sulfonyl; carboxyl; hydroxyl; C1-12 unsubstituted, substituted, linear or branched alkyl, alkenyl or alkynyl; amino; amido; aryl, halo, alkoxy, nitro, and further wherein two adjacent R groups may be taken together to form an aryl, heteroaryl, cycloalkyl or cycloheteroaryl group.

47. A compound or salt thereof having a formula selected from: 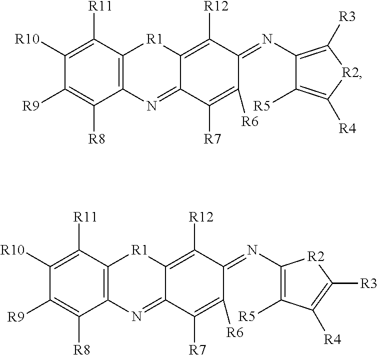
wherein R1 is either O or S, R2 is either O, S or NH and R3-12 may be the same or different and may be independently selected from the group comprising hydrogen; sulfonyl; carboxyl; hydroxyl; C1-12 unsubstituted, substituted, linear or branched alkyl, alkenyl or alkynyl; amino; amido; aryl, halo, alkoxy, nitro, and further wherein two adjacent R groups may be taken together to form an aryl, heteroaryl, cycloalkyl or cycloheteroaryl group.

48. A compound or salt thereof having the formula: 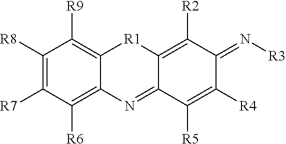
wherein R1 is S or O, R3 is either H, CH₂OOH, CH₂SO₃H, CH₂NH₂ or CH₂NO₂, and R2 and R4-R9 may be the same or different and may be independently selected from the group comprising hydrogen; sulfonyl; carboxyl; hydroxyl; C1-12 unsubstituted, substituted, linear or branched alkyl, alkenyl or alkynyl; amino; amido; aryl, halo, alkoxy, nitro, and further wherein two adjacent R groups may be taken together to form an aryl, heteroaryl, cycloalkyl or cycloheteroaryl group.

49. A compound or salt thereof selected from the group consisting of:

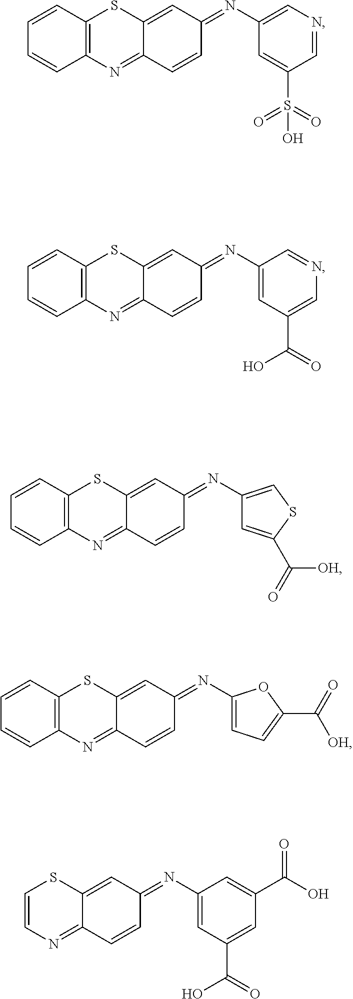 [SMILES: O=S(=O)(O)c1cncc(/N=c2\ccc3nc4ccccc4sc-3c2)c1 ; O=C(O)c1cncc(/N=c2\ccc3nc4ccccc4sc-3c2)c1 ; O=C(O)c1cc(/N=c2\ccc3nc4ccccc4sc-3c2)cs1 ; O=C(O)c1ccc(/N=c2\ccc3nc4ccccc4sc-3c2)o1 ; O=C(O)c1cc(/N=c2\ccc3nccsc-3c2)cc(C(=O)O)c1]

50. Use of the compound or salt thereof of any one of claims 42-49 as a component of a reagent composition in an electrochemical assay comprising an oxidoreductase enzyme and a buffer salt.

51. Use according to claim 50, wherein the electrochemical assay is any electrochemical immunoassay comprising an antibody conjugated with the oxidoreductase enzyme, and wherein the oxidoreductase enzyme converts the compound or salt thereof from an oxidised form to a reduced form, and wherein an electrode is used to convert the reduced compound or salt thereof back to an oxidised form, in so doing transferring at least one electron to the electrode which is recorded as an electrical current.

52. A mixture comprising the compound or salt thereof of any one of claims 42-49 and a biological fluid sample, wherein the fluid is selected from blood, plasma, serum, cerebrospinal fluid, urine, saliva, sputum, semen.
